(19) United States
(12) Patent Application Publication
(10) Pub. No.: US 2026/0014348 A1
(43) Pub. Date: Jan. 15, 2026

(54) MEDICAL DEVICE PACKAGING

(71) Applicant: ConvaTec Limited, Flintshire (GB)

(72) Inventor: Vladimir Jenco, Michalovce (SK)

(21) Appl. No.: 19/330,061

(22) Filed: Sep. 16, 2025

Related U.S. Application Data
(63) Continuation of application No. PCT/GB2024/050614, filed on Mar. 8, 2024.
(60) Provisional application No. 63/452,868, filed on Mar. 17, 2023.

(30) Foreign Application Priority Data
Mar. 30, 2023 (GB) 2304719.4

(51) Int. Cl. A61M 25/00

(52) U.S. Cl. CPC A61M 25/002; A61M 2207/00; A61M 2209/06

(57) ABSTRACT

An intermittent catheter pouch for storing an intermittent catheter, the pouch comprising: a first polymer film and a second polymer film, wherein the first and second polymer films are sealed together at a sealing region to form a cavity therebetween, wherein the first and second films each have an inner surface that faces the inner surface of the other film and an outer surface that faces the exterior of the pouch, wherein the inner surface of the first film and the inner surface of the second film comprise a non-oriented polyolefin, and the outer surface of the first film and the outer surface of the second film comprise an oriented polyolefin, and wherein at least a portion of the sealing region is peelable to form an opening in the pouch.

### TECHNICAL FIELD OF THE INVENTION

The present invention relates to medical device packaging and methods for manufacturing such packaging.

### BACKGROUND TO THE INVENTION

Medical devices need to be maintained in a sterile environment to avoid contamination and prevent infection. Therefore, it is important that packaging for medical devices is well-sealed to maintain the sterility of the contents inside. This is especially important for urinary catheters, as urinary tract infections (UTIs) and other infections are common, and the catheter must be kept as sterile and as possible to avoid contamination and reduce the risk of infection. It is also common for urinary catheters to packaged with a wetting or lubricating agent, which adds an additional level of complexity to the packaging, as it must be both leak-proof and capable of holding its contents without compromising sterility.

Unfortunately, current packaging options that meet these requirements can be challenging to open, with releasable portions of the packaging often sticking and requiring considerable force and effort to manipulate. This can be especially problematic for catheters used by patients who practice self-catheterisation and may have limited dexterity, resulting in an increased risk of contamination during the opening process.

Current packaging materials used have also been found to suffer from the disadvantage of the packaging material weakening or being negatively impacted during sealing of the packaging. This can lead to the packaging tearing easily, which can compromise the sterility of the contained medical device, and it can also make it difficult to open the packaging when needed. It is an aim of embodiments of the present invention to address one or more of the above problems by providing medical device packaging that is well-sealed when closed, preferably allowing the sterility of the contents to be maintained, whilst also being easy to open.

It is also an aim of embodiments of the present invention to overcome or mitigate at least one problem of the prior art, whether expressly described herein or not.

### SUMMARY OF THE INVENTION

According to a first aspect of the invention, there is provided a container for storing a medical device, the container comprising: a first polymer film and a second polymer film, wherein the first and second polymer films are sealed together at a sealing region to form a cavity therebetween, wherein the first and second films each have an inner surface that faces the inner surface of the other film and an outer surface that faces the exterior of the container, wherein the inner surface of the first film and the inner surface of the second film comprise a non-oriented polyolefin, and the outer surface of the first film and the outer surface of the second film comprise an oriented polyolefin, and wherein at least a portion of the sealing region is peelable to form an opening in the container.

According to a second aspect of the invention, there is provided an intermittent catheter pouch for storing an intermittent catheter, the pouch comprising: a first polymer film and a second polymer film, wherein the first and second polymer films are sealed together at a sealing region to form a cavity therebetween, wherein the first and second films each have an inner surface that faces the inner surface of the other film and an outer surface that faces the exterior of the pouch, wherein the inner surface of the first film and the inner surface of the second film comprise a non-oriented polyolefin, and the outer surface of the first film and the outer surface of the second film comprise an oriented polyolefin, and wherein at least a portion of the sealing region is peelable to form an opening in the pouch.

The combination of the non-oriented polyolefin inner surfaces and oriented polyolefin outer surfaces of the first and second films provides for optimal sealing conditions, which allows for a strong and effective seal to be formed, allowing for the sterility of the contents of the container to be maintained when closed. Moreover, such film materials also allow for a peelable portion to be provided that is easy to peel, without the films sticking and without requiring a large amount of force to be imparted.

The film materials also allow for selective sealing together of the inner surfaces of the films. The outer surfaces are minimally impacted by the sealing process. The result is a container with optimal mechanical properties and which suffers minimally from undesired tearing.

Overall, the combination of these film materials and their properties enables the provision of a container that both provides a sterile internal environment and that is easy to open, and remain open, whilst maintaining minimal packaging material (two films) making it well-suited for storing medical devices, particularly catheters, in a safe and accessible manner.

The following statements apply mutatis mutandis to the first and second aspects of the invention.

By “polyolefin”, reference is made to any polymer or copolymer produced from an olefin or alkene monomer.

In statements of invention below, reference to “the oriented polyolefin” may refer to the oriented polyolefin of the first film, the oriented polyolefin of the second film, or both. In preferred embodiments, the oriented polyolefin of the first film and the oriented polyolefin of the second film comprise the same polyolefin. In some embodiments, the oriented polyolefin of the first film is the oriented polyolefin of the second film. In other embodiments, the oriented polyolefin of the first film is different to the oriented polyolefin of the second film.

In statements of invention below, reference to “the non-oriented polyolefin” may refer to the non-oriented polyolefin of the first film, the non-oriented polyolefin of the second film, or both. In preferred embodiments, the oriented polyolefin of the first film and the oriented polyolefin of the second film comprise the same polyolefin. In some embodiments, the non-oriented polyolefin of the first film is the non-oriented polyolefin of the second film. In other embodiments, the non-oriented polyolefin of the first film is different to the non-oriented polyolefin of the second film.

In some embodiments, the oriented polyolefin and/or the non-oriented polyolefin is a thermoplastic polyolefin.

In some embodiments, the oriented polyolefin and/or the non-oriented polyolefin is independently chosen from: a low-density polyolefin, a linear low-density polyolefin, a high-density polyolefin, an ultra-high-molecular weight polyolefin, a cross-linked polyolefin, a medium-density polyolefin, and combinations thereof. In some embodiments, the oriented polyolefin and/or the non-oriented polyolefin is independently selected from the group consisting of: a low-density polyolefin, a linear low-density polyolefin, a high-density polyolefin, an ultra-high-molecular weight polyolefin, a cross-linked polyolefin, a medium-density polyolefin, and combinations thereof.

In some embodiments, the oriented polyolefin and/or the non-oriented polyolefin is a low-density polyolefin. In preferred embodiments, only the non-oriented polyolefin is a low-density polyolefin.

In some preferred embodiments, there is provided a container for storing a medical device, the container comprising: a first polymer film and a second polymer film, wherein the first and second polymer films are sealed together at a sealing region to form a cavity therebetween, wherein the first and second films each have an inner surface that faces the inner surface of the other film and an outer surface that faces the exterior of the container, wherein the inner surface of the first film and the inner surface of the second film comprise a non-oriented polyolefin, and the outer surface of the first film and the outer surface of the second film comprise an oriented polyolefin, and wherein at least a portion of the sealing region is peelable to form an opening in the container, and wherein the oriented polyolefin and/or the non-oriented polyolefin is a low-density polyolefin.

In some preferred embodiments, there is provided an intermittent catheter pouch for storing an intermittent catheter, the pouch comprising: a first polymer film and a second polymer film, wherein the first and second polymer films are sealed together at a sealing region to form a cavity therebetween, wherein the first and second films each have an inner surface that faces the inner surface of the other film and an outer surface that faces the exterior of the pouch, wherein the inner surface of the first film and the inner surface of the second film comprise a non-oriented polyolefin, and the outer surface of the first film and the outer surface of the second film comprise an oriented polyolefin, and wherein at least a portion of the sealing region is peelable to form an opening in the pouch, and wherein the oriented polyolefin and/or the non-oriented polyolefin is a low-density polyolefin.

In some preferred embodiments, there is provided a container for storing a medical device, the container comprising: a first polymer film and a second polymer film, wherein the first and second polymer films are sealed together at a sealing region to form a cavity therebetween, wherein the first and second films each have an inner surface that faces the inner surface of the other film and an outer surface that faces the exterior of the container, wherein the inner surface of the first film and the inner surface of the second film comprise a non-oriented polyolefin, and the outer surface of the first film and the outer surface of the second film comprise an oriented polyolefin, and wherein at least a portion of the sealing region is peelable to form an opening in the container, and wherein only the non-oriented polyolefin is a low-density polyolefin.

In some preferred embodiments, there is provided an intermittent catheter pouch for storing an intermittent catheter, the pouch comprising: a first polymer film and a second polymer film, wherein the first and second polymer films are sealed together at a sealing region to form a cavity therebetween, wherein the first and second films each have an inner surface that faces the inner surface of the other film and an outer surface that faces the exterior of the pouch, wherein the inner surface of the first film and the inner surface of the second film comprise a non-oriented polyolefin, and the outer surface of the first film and the outer surface of the second film comprise an oriented polyolefin, and wherein at least a portion of the sealing region is peelable to form an opening in the pouch, and wherein only the non-oriented polyolefin is a low-density polyolefin.

In some embodiments, the oriented polyolefin and/or the non-oriented polyolefin is a linear low-density polyolefin. In preferred embodiments, only the non-oriented polyolefin is a linear low-density polyolefin.

In some preferred embodiments, there is provided a container for storing a medical device, the container comprising: a first polymer film and a second polymer film, wherein the first and second polymer films are sealed together at a sealing region to form a cavity therebetween, wherein the first and second films each have an inner surface that faces the inner surface of the other film and an outer surface that faces the exterior of the container, wherein the inner surface of the first film and the inner surface of the second film comprise a non-oriented polyolefin, and the outer surface of the first film and the outer surface of the second film comprise an oriented polyolefin, and wherein at least a portion of the sealing region is peelable to form an opening in the container, and wherein the oriented polyolefin and/or the non-oriented polyolefin is a linear low-density polyolefin.

In some preferred embodiments, there is provided an intermittent catheter pouch for storing an intermittent catheter, the pouch comprising: a first polymer film and a second polymer film, wherein the first and second polymer films are sealed together at a sealing region to form a cavity therebetween, wherein the first and second films each have an inner surface that faces the inner surface of the other film and an outer surface that faces the exterior of the pouch, wherein the inner surface of the first film and the inner surface of the second film comprise a non-oriented polyolefin, and the outer surface of the first film and the outer surface of the second film comprise an oriented polyolefin, and wherein at least a portion of the sealing region is peelable to form an opening in the pouch, and wherein the oriented polyolefin and/or the non-oriented polyolefin is a linear low-density polyolefin.

In some preferred embodiments, there is provided a container for storing a medical device, the container comprising: a first polymer film and a second polymer film, wherein the first and second polymer films are sealed together at a sealing region to form a cavity therebetween, wherein the first and second films each have an inner surface that faces the inner surface of the other film and an outer surface that faces the exterior of the container, wherein the inner surface of the first film and the inner surface of the second film comprise a non-oriented polyolefin, and the outer surface of the first film and the outer surface of the second film comprise an oriented polyolefin, and wherein at least a portion of the sealing region is peelable to form an opening in the container, and wherein only the non-oriented polyolefin is a linear low-density polyolefin.

In some preferred embodiments, there is provided an intermittent catheter pouch for storing an intermittent catheter, the pouch comprising: a first polymer film and a second polymer film, wherein the first and second polymer films are sealed together at a sealing region to form a cavity therebetween, wherein the first and second films each have an inner surface that faces the inner surface of the other film and an outer surface that faces the exterior of the pouch, wherein the inner surface of the first film and the inner surface of the second film comprise a non-oriented polyolefin, and the outer surface of the first film and the outer surface of the second film comprise an oriented polyolefin, and wherein at least a portion of the sealing region is peelable to form an opening in the pouch, and wherein only the non-oriented polyolefin is a linear low-density polyolefin.

In some embodiments, the oriented polyolefin and/or the non-oriented polyolefin is independently chosen from: polyethylene, polypropylene, ethylene-vinyl acetate, polybutene, an ethylene-alpha olefin copolymer, ethylene methyl acrylate, polyvinyl alcohol, polyvinyl chloride, polyvinylidene chloride, ethylene vinyl alcohol, acrylonitrile-butadiene-styrene, poly(methyl methacrylate), polystyrene, polymethylpentene, polyvinyl acetate, polyacrylamide, polyacrylate, ethylene/methacrylate copolymer, ethylene/n-butyl acrylate, ethylene/vinyl acetate/carbon monoxide, ethylene/acrylic acid, ethylene/methacrylic acid, and combinations, blends or copolymers thereof. In some embodiments, the oriented polyolefin and/or the non-oriented polyolefin is independently selected from the group consisting of: polyethylene, polypropylene, ethylene-vinyl acetate, polybutene, an ethylene-alpha olefin copolymer, ethylene methyl acrylate, polyvinyl alcohol, polyvinyl chloride, polyvinylidene chloride, ethylene vinyl alcohol, acrylonitrile-butadiene-styrene, poly(methyl methacrylate), polystyrene, polymethylpentene, polyvinyl acetate, polyacrylamide, polyacrylate, ethylene/methacrylate copolymer, ethylene/n-butyl acrylate, ethylene/vinyl acetate/carbon monoxide, ethylene/acrylic acid, ethylene/methacrylic acid, and combinations, blends or copolymers thereof.

In preferred embodiments, the oriented polyolefin and/or the non-oriented polyolefin is independently chosen from: polyethylene, polypropylene, and combinations, blends or copolymers thereof. In preferred embodiments, the oriented polyolefin and/or the non-oriented polyolefin is independently selected from the group consisting of: polyethylene, polypropylene, and combinations, blends or copolymers thereof.

In particularly preferred embodiments, the oriented polyolefin and/or the non-oriented polyolefin is polyethylene.

In some preferred embodiments, there is provided a container for storing a medical device, the container comprising: a first polymer film and a second polymer film, wherein the first and second polymer films are sealed together at a sealing region to form a cavity therebetween, wherein the first and second films each have an inner surface that faces the inner surface of the other film and an outer surface that faces the exterior of the container, wherein the inner surface of the first film and the inner surface of the second film comprise a non-oriented polyolefin, and the outer surface of the first film and the outer surface of the second film comprise an oriented polyolefin, and wherein at least a portion of the sealing region is peelable to form an opening in the container, and wherein the oriented polyolefin and/or the non-oriented polyolefin is polyethylene.

In some preferred embodiments, there is provided an intermittent catheter pouch for storing an intermittent catheter, the pouch comprising: a first polymer film and a second polymer film, wherein the first and second polymer films are sealed together at a sealing region to form a cavity therebetween, wherein the first and second films each have an inner surface that faces the inner surface of the other film and an outer surface that faces the exterior of the pouch, wherein the inner surface of the first film and the inner surface of the second film comprise a non-oriented polyolefin, and the outer surface of the first film and the outer surface of the second film comprise an oriented polyolefin, and wherein at least a portion of the sealing region is peelable to form an opening in the pouch, and wherein the oriented polyolefin and/or the non-oriented polyolefin is polyethylene.

In preferred embodiments, the oriented polyolefin and the non-oriented polyolefin comprise the same polyolefin. In such embodiments, the outer surface of the first film may comprise the same polyolefin as the inner surface of the first film. In such embodiments, the outer surface of the second film may comprise the same polyolefin as the inner surface of the second film. Preferably, the oriented polyolefin and the non-oriented polyolefin are both polyethylene. In other embodiments, the oriented polyolefin and the non-oriented polyolefin comprise different polyolefins. In such embodiments, the oriented or non-oriented polyolefin may be polyethylene, and the other polyolefin may be a different polyolefin, which may be polypropylene.

In some preferred embodiments, there is provided a container for storing a medical device, the container comprising: a first polymer film and a second polymer film, wherein the first and second polymer films are sealed together at a sealing region to form a cavity therebetween, wherein the first and second films each have an inner surface that faces the inner surface of the other film and an outer surface that faces the exterior of the container, wherein the inner surface of the first film and the inner surface of the second film comprise a non-oriented polyolefin, and the outer surface of the first film and the outer surface of the second film comprise an oriented polyolefin, and wherein at least a portion of the sealing region is peelable to form an opening in the container, and wherein the oriented polyolefin and the non-oriented polyolefin comprise the same polyolefin.

In some preferred embodiments, there is provided an intermittent catheter pouch for storing an intermittent catheter, the pouch comprising: a first polymer film and a second polymer film, wherein the first and second polymer films are sealed together at a sealing region to form a cavity therebetween, wherein the first and second films each have an inner surface that faces the inner surface of the other film and an outer surface that faces the exterior of the pouch, wherein the inner surface of the first film and the inner surface of the second film comprise a non-oriented polyolefin, and the outer surface of the first film and the outer surface of the second film comprise an oriented polyolefin, and wherein at least a portion of the sealing region is peelable to form an opening in the pouch, and wherein the oriented polyolefin and the non-oriented polyolefin comprise the same polyolefin.

In preferred embodiments, the non-oriented polyolefin of the first film comprises the same polyolefin as the non-oriented polyolefin of the second film. In some embodiments, the non-oriented polyolefin of the first film is the non-oriented polyolefin of the second film. In such embodiments, the inner surface of the first film may comprise the same polyolefin as the inner surface of the second film. Preferably, the inner surfaces of the first and second films comprise polyethylene, preferably linear low-density polyethylene. In other embodiments, the inner surfaces of the first and second films may comprise different polyolefins. In such embodiments, the inner surface of one film may comprise polyethylene and the inner surface of the second film may comprise a different polyolefin, which may be polypropylene.

In preferred embodiments, the oriented polyolefin of the first film comprises the same polyolefin as the oriented polyolefin of the second film. In some embodiments, the oriented polyolefin of the first film is the oriented polyolefin of the second film. In such embodiments, the outer surface of the first film may comprise the same polyolefin as the outer surface of the second film. Preferably, the outer surfaces of the first and second films comprise polyethylene, preferably biaxially oriented polyethylene. In other embodiments, the outer surfaces of the first and second films may comprise different polyolefins. In such embodiments, the outer surface of one film may comprise polyethylene and the outer surface of the second film may comprise a different polyolefin, which may be polypropylene.

In some embodiments, the inner surfaces of the first and second films and the outer surfaces of the first and second films comprise the same polyolefin. The inner surfaces of the first and second films and the outer surfaces of the first and second films may preferably all comprise polyethylene.

In some embodiments, the first film is made of a single polyolefin. The first film may be made of a polyolefin independently chosen from: polyethylene, polypropylene, and combinations, blends or copolymers thereof. The first film may be made of a polyolefin independently selected from the group consisting of: polyethylene, polypropylene, and combinations, blends or copolymers thereof. The first film may preferably be made of polyethylene. In some embodiments, the second film is made of a single polyolefin. The second film may be made of a polyolefin independently chosen from: polyethylene, polypropylene, and combinations, blends or copolymers thereof. The second film may be made of a polyolefin independently selected from the group consisting of: polyethylene, polypropylene, and combinations, blends or copolymers thereof. The second film may preferably be made of polyethylene. In some embodiments, the first film is made of the same polyolefin as the second film. In preferred embodiments, the first and second films are made of polyethylene. In other embodiments, the first film and second film are made of different polyolefins. In such embodiments, the first or second film may be made of polyethylene, and the other film may be made of a different polyolefin, which may be polypropylene.

In some preferred embodiments, there is provided a container for storing a medical device, the container comprising: a first polymer film and a second polymer film, wherein the first and second polymer films are sealed together at a sealing region to form a cavity therebetween, wherein the first and second films each have an inner surface that faces the inner surface of the other film and an outer surface that faces the exterior of the container, wherein the inner surface of the first film and the inner surface of the second film comprise a non-oriented polyolefin, and the outer surface of the first film and the outer surface of the second film comprise an oriented polyolefin, and wherein at least a portion of the sealing region is peelable to form an opening in the container, and wherein the first and/or second film is made of a single polyolefin.

In some preferred embodiments, there is provided an intermittent catheter pouch for storing an intermittent catheter, the pouch comprising: a first polymer film and a second polymer film, wherein the first and second polymer films are sealed together at a sealing region to form a cavity therebetween, wherein the first and second films each have an inner surface that faces the inner surface of the other film and an outer surface that faces the exterior of the pouch, wherein the inner surface of the first film and the inner surface of the second film comprise a non-oriented polyolefin, and the outer surface of the first film and the outer surface of the second film comprise an oriented polyolefin, and wherein at least a portion of the sealing region is peelable to form an opening in the pouch, and wherein the first and/or second film is made of a single polyolefin.

In some embodiments, the oriented polyolefin and/or the non-oriented polyolefin is low-density polyethylene. In preferred embodiments, only the non-oriented polyolefin is low-density polyethylene.

In some preferred embodiments, there is provided a container for storing a medical device, the container comprising: a first polymer film and a second polymer film, wherein the first and second polymer films are sealed together at a sealing region to form a cavity therebetween, wherein the first and second films each have an inner surface that faces the inner surface of the other film and an outer surface that faces the exterior of the container, wherein the inner surface of the first film and the inner surface of the second film comprise a non-oriented polyolefin, and the outer surface of the first film and the outer surface of the second film comprise an oriented polyolefin, and wherein at least a portion of the sealing region is peelable to form an opening in the container, and wherein the oriented polyolefin and/or the non-oriented polyolefin is low-density polyethylene.

In some preferred embodiments, there is provided an intermittent catheter pouch for storing an intermittent catheter, the pouch comprising: a first polymer film and a second polymer film, wherein the first and second polymer films are sealed together at a sealing region to form a cavity therebetween, wherein the first and second films each have an inner surface that faces the inner surface of the other film and an outer surface that faces the exterior of the pouch, wherein the inner surface of the first film and the inner surface of the second film comprise a non-oriented polyolefin, and the outer surface of the first film and the outer surface of the second film comprise an oriented polyolefin, and wherein at least a portion of the sealing region is peelable to form an opening in the pouch, and wherein the oriented polyolefin and/or the non-oriented polyolefin is low-density polyethylene.

In some preferred embodiments, there is provided a container for storing a medical device, the container comprising: a first polymer film and a second polymer film, wherein the first and second polymer films are sealed together at a sealing region to form a cavity therebetween, wherein the first and second films each have an inner surface that faces the inner surface of the other film and an outer surface that faces the exterior of the container, wherein the inner surface of the first film and the inner surface of the second film comprise a non-oriented polyolefin, and the outer surface of the first film and the outer surface of the second film comprise an oriented polyolefin, and wherein at least a portion of the sealing region is peelable to form an opening in the container, and wherein only the non-oriented polyolefin is low-density polyethylene.

In some preferred embodiments, there is provided an intermittent catheter pouch for storing an intermittent catheter, the pouch comprising: a first polymer film and a second polymer film, wherein the first and second polymer films are sealed together at a sealing region to form a cavity therebetween, wherein the first and second films each have an inner surface that faces the inner surface of the other film and an outer surface that faces the exterior of the pouch, wherein the inner surface of the first film and the inner surface of the second film comprise a non-oriented polyolefin, and the outer surface of the first film and the outer surface of the second film comprise an oriented polyolefin, and wherein at least a portion of the sealing region is peelable to form an opening in the pouch, and wherein only the non-oriented polyolefin is low-density polyethylene.

In some embodiments, the oriented polyolefin and/or the non-oriented polyolefin is linear low-density polyethylene. In preferred embodiments, only the non-oriented polyolefin is linear low-density polyethylene.

In some preferred embodiments, there is provided a container for storing a medical device, the container comprising: a first polymer film and a second polymer film, wherein the first and second polymer films are sealed together at a sealing region to form a cavity therebetween, wherein the first and second films each have an inner surface that faces the inner surface of the other film and an outer surface that faces the exterior of the container, wherein the inner surface of the first film and the inner surface of the second film comprise a non-oriented polyolefin, and the outer surface of the first film and the outer surface of the second film comprise an oriented polyolefin, and wherein at least a portion of the sealing region is peelable to form an opening in the container, and wherein the oriented polyolefin and/or the non-oriented polyolefin is linear low-density polyethylene.

In some preferred embodiments, there is provided an intermittent catheter pouch for storing an intermittent catheter, the pouch comprising: a first polymer film and a second polymer film, wherein the first and second polymer films are sealed together at a sealing region to form a cavity therebetween, wherein the first and second films each have an inner surface that faces the inner surface of the other film and an outer surface that faces the exterior of the pouch, wherein the inner surface of the first film and the inner surface of the second film comprise a non-oriented polyolefin, and the outer surface of the first film and the outer surface of the second film comprise an oriented polyolefin, and wherein at least a portion of the sealing region is peelable to form an opening in the pouch, and wherein the oriented polyolefin and/or the non-oriented polyolefin is linear low-density polyethylene.

In some preferred embodiments, there is provided a container for storing a medical device, the container comprising: a first polymer film and a second polymer film, wherein the first and second polymer films are sealed together at a sealing region to form a cavity therebetween, wherein the first and second films each have an inner surface that faces the inner surface of the other film and an outer surface that faces the exterior of the container, wherein the inner surface of the first film and the inner surface of the second film comprise a non-oriented polyolefin, and the outer surface of the first film and the outer surface of the second film comprise an oriented polyolefin, and wherein at least a portion of the sealing region is peelable to form an opening in the container, and wherein only the non-oriented polyolefin is linear low-density polyethylene.

In some preferred embodiments, there is provided an intermittent catheter pouch for storing an intermittent catheter, the pouch comprising: a first polymer film and a second polymer film, wherein the first and second polymer films are sealed together at a sealing region to form a cavity therebetween, wherein the first and second films each have an inner surface that faces the inner surface of the other film and an outer surface that faces the exterior of the pouch, wherein the inner surface of the first film and the inner surface of the second film comprise a non-oriented polyolefin, and the outer surface of the first film and the outer surface of the second film comprise an oriented polyolefin, and wherein at least a portion of the sealing region is peelable to form an opening in the pouch, and wherein only the non-oriented polyolefin is linear low-density polyethylene.

In some embodiments, the oriented polyolefin is independently chosen from: a monoaxially oriented polyolefin, a biaxially oriented polyolefin, an oriented high-density polyolefin, an oriented ultra-high-molecular-weight polyolefin, a machine direction oriented polyolefin, and a tenter frame oriented polyolefin. In some embodiments, the oriented polyolefin is independently selected from the group consisting of: a monoaxially oriented polyolefin, a biaxially oriented polyolefin, an oriented high-density polyolefin, an oriented ultra-high-molecular-weight polyolefin, a machine direction oriented polyolefin, and a tenter frame oriented polyolefin. In some embodiments, the oriented polyolefin is monoaxially oriented. In particularly preferred embodiments, the oriented polyolefin is biaxially oriented. In such embodiments, the oriented polyolefin may be independently chosen from: polyethylene, polypropylene, and combinations, blends or copolymers thereof. In such embodiments, the oriented polyolefin may be independently selected from the group consisting of: polyethylene, polypropylene, and combinations, blends or copolymers thereof. In preferred embodiments, the oriented polyolefin is polyethylene.

In some embodiments, the oriented polyolefin is monoaxially oriented or biaxially oriented, and the non-oriented polyolefin is a low-density polyolefin. In some embodiments, the oriented polyolefin is monoaxially oriented or biaxially oriented, and the non-oriented polyolefin is a linear low-density polyolefin. In some preferred embodiments, the oriented polyolefin is biaxially oriented, and the non-oriented polyolefin is a linear low-density polyolefin. In such embodiments, the oriented polyolefin and the non-oriented polyolefin preferably comprise the same polyolefin. In such embodiments, the oriented polyolefin and the non-oriented polyolefin may be independently chosen from: polyethylene, polypropylene, and combinations, blends or copolymers thereof. In such embodiments, the oriented polyolefin and the non-oriented polyolefin may be independently selected from the group consisting of: polyethylene, polypropylene, and combinations, blends or copolymers thereof. In such embodiments, the oriented polyolefin and the non-oriented polyolefin both comprise polyethylene. In particularly preferred embodiments, the oriented polyolefin is biaxially oriented polyethylene and the non-oriented polyolefin is linear low-density polyethylene.

The inner surface of the first and/or second film preferably comprises the non-oriented polyolefin on at least the portion of the inner surface area that comprises the peelable portion of the sealing region. The inner surface of the first and/or second film may comprise the non-oriented polyolefin on at least the portion of the inner surface area that comprises the entire sealing region. The inner surface of the first and/or second film preferably comprises the non-oriented polyolefin on at least the portion of the inner surface area that does not include the peelable portion of the sealing region. The inner surface of the first and/or second film may comprise the non-oriented polyolefin on the entire inner surface area of the film.

The outer surface of the first and/or second film preferably comprises the oriented polyolefin on at least the portion of the outer surface area that comprises the peelable portion of the sealing region. The outer surface of the first and/or second film may comprise the oriented polyolefin on at least the portion of the outer surface area that comprises the entire sealing region. The outer surface of the first and/or second film preferably comprises the oriented polyolefin on at least the portion of the outer surface area that does not include the peelable portion of the sealing region. The outer surface of the first and/or second film may comprise the oriented polyolefin on the entire outer surface area of the film.

In some embodiments, the first and/or second film is made from a liquid impermeable material. The first and/or second film may be made of a liquid and gas impermeable material.

In some embodiments, the first film and/or the second film is a laminate film formed of at least two polymer layers. The laminate film may be formed of a plurality of layers. The laminate film may comprise 2, 3, 4, or 5 polymer layers.

The polymer layers of the laminate film may comprise stacked layers that are laminated together.

In some embodiments, the polymer layers of the laminate film may be laminated by a method that is independently chosen from: adhesive bonding, extrusion lamination, solvent-based lamination, heat sealing, and pressure-sensitive adhesive bonding. In some embodiments, the polymer layers of the laminate film may be laminated by a method that is independently selected from the group consisting of: adhesive bonding, extrusion lamination, solvent-based lamination, heat sealing, and pressure-sensitive adhesive bonding. The polymer layers of the laminate film may be laminated by an adhesive bond. The adhesive bond may comprise an adhesive layer between the laminated polymer layers.

At least the inner surface of the laminate film may comprise or be made of a non-oriented polyolefin. At least the outer surface of the laminate film may comprise or be made of an oriented polyolefin. In some embodiments, the inner and outer surfaces of the film comprise different layers of the laminate film. In some embodiments, the laminate film comprises at least one further polymer layer between the layer comprising the outer surface of the film and the layer comprising the inner surface of the film. At least one further polymer layer may be an adhesive layer.

In some embodiments, the film comprises two polymer layers, the inner surface of the film comprising one layer and the outer surface of the film comprising the other layer. In such embodiments, the layer comprising the inner surface of the film may be in direct contact with the layer comprising the outer surface of the film. The layer comprising the inner surface of the film may be laminated directly to the layer comprising the outer surface of the film. In some embodiments, the first and second films both comprise the same laminate film. In some embodiments, the inner surface of the first laminate film comprises or is made of a non-oriented polyolefin, and the outer surface of the first laminate film comprises or is made of an oriented polyolefin. In some embodiments, the inner surface of the second laminate film comprises or is made of a non-oriented polyolefin, and the outer surface of the second laminate film comprises or is made of an oriented polyolefin. In some embodiments, the oriented polyolefin is monoaxially oriented or biaxially oriented, and the non-oriented polyolefin is a low-density polyolefin. In some embodiments, the oriented polyolefin is monoaxially oriented or biaxially oriented, and the non-oriented polyolefin is a linear low-density polyolefin. In some preferred embodiments, the oriented polyolefin is biaxially oriented, and the non-oriented polyolefin is a linear low-density polyolefin. In such embodiments, the oriented polyolefin and the non-oriented polyolefin preferably comprise the same polyolefin. In such embodiments, the oriented polyolefin and the non-oriented polyolefin may be independently chosen from: polyethylene, polypropylene, and combinations, blends or copolymers thereof. In such embodiments, the oriented polyolefin and the non-oriented polyolefin may be independently selected from the group consisting of: polyethylene, polypropylene, and combinations, blends or copolymers thereof. In such embodiments, the oriented polyolefin and the non-oriented polyolefin both comprise polyethylene. In particularly preferred embodiments, the oriented polyolefin is biaxially oriented polyethylene and the non-oriented polyolefin is linear low-density polyethylene.

In some embodiments, the first and/or second film each has a thickness of at least 60 microns, or at least 65, 70, 75, or at least 80 microns, or at least 85, 90, 95, or at least 100 microns. The first and/or second film may each have a thickness of no greater than 150 microns, or no greater than 145, 140, 135, or no greater than 130 microns. In some embodiments, the first and/or second film each has a thickness of between 60-150 microns, or between 100-130 microns, or between 105-125, or between 110-120 microns.

In some embodiments, the first and/or second film may have a seal initiation temperature of at least 70° C., or at least 75, 80, 85, or at least 90° C. The first and/or second film may have a seal initiation temperature of no greater than 180°, or no greater than 175, 170, 165, 160, 155, 150, or no greater than 145° C. The first and/or second film may have a seal initiation temperature of between 70-165° C., or between 75-160, 80-155, 85-150, or between 90-145° C.

In some embodiments, the first and/or second film has a surface density of at least 70 g/m², or at least 75, 80, 85, 90, or at least 95 g/m². The first and/or second film may have a surface density of no greater than 150 g/m², or no greater than 145, 140, 135, 130, 125, or no greater than 120 g/m². The first and/or second film may have a surface density of between 85-125 g/m², or between 90-120, 95-115, or between 100-110 g/m².

In some embodiments, the first and/or second film has a water vapour transmission of no greater than 7 g/m²/day, or no greater than 6.5, 6, 5.5, 5, 4.5, 4, 3.5, 3, 2.5, or no greater than 2 g/m²/day.

In some embodiments, the first and/or second film has an oxygen transmission of no greater than 3 cm³/m²/day/atm, or no greater than 2.5, 2, 1.5, or no greater than 1 cm³/m²/day/atm.

In some embodiments, the inner surface of the first and/or second film has a coefficient of friction of at least 0.025, 0.05, 0.075, 0.1, 0.15, or at least 0.2. The inner surface of the first and/or second film may have a coefficient of friction of no greater than 0.8, or no greater than 0.75, 0.7, 0.65, 0.6, 0.55, or no greater than 0.5. The inner surface of the first and/or second film may have a coefficient of friction of between 0.05-0.65, or between 0.1-0.6, 0.15-0.55, or between 0.2-0.5.

In some embodiments, the peelable portion of the sealing region has a peel strength of at least 1.2 N/15 mm, or at least 1.3, 1.4, 1.5, 2, 2.5, or at least 3 N/15 mm. The peelable portion of the sealing region may have a peel strength of no greater than 10 N/15 mm, or no greater than 9.5, 9, 8.5, or no greater than 8 N/15 mm. The peelable portion of the sealing region may have a peel strength of between 1.2-10 N/15 mm, or between 1.5-9.5, 2-9, 2.5-8.5, or between 3-8 N/15 mm.

In some embodiments, the first and/or second film has a shape independently chosen from: a rectangle, a square, a circle, an oval, and a trapezoid. In some embodiments, the first and/or second film has a shape independently selected from the group consisting of: a rectangle, a square, a circle, an oval, and a trapezoid. One with skill in the art will recognize that the first and second films may take a range of other shapes.

In some embodiments, the first and/or second film comprises a top edge, a bottom edge, and opposed first and second side edges.

In some embodiments, the first and second films are sealed together at the sealing region to form a gas-tight and/or vapour-tight cavity therebetween. The first and second films are preferably sealed together at the sealing region to maintain a sterile internal environment.

In some embodiments, the peelable portion of the sealing region between the first and second films comprises a seal that is independently chosen from: a heat seal, a cold seal, an adhesive seal, a cohesive seal, a weld seal, an ultrasonic seal, a hot melt seal, a pressure-sensitive seal, and a seal created by a micro-embossed structure. In some embodiments, the peelable portion of the sealing region between the first and second films comprises a seal that is independently selected from the group consisting of: a heat seal, a cold seal, an adhesive seal, a cohesive seal, a weld seal, an ultrasonic seal, a hot melt seal, a pressure-sensitive seal, and a seal created by a micro-embossed structure. In some embodiments, the peelable portion of the sealing region between the first and second films comprises a heat seal.

In some embodiments, the entire sealing region comprises a peelable seal. In other embodiments, a portion of the sealing region comprises a peelable seal and another portion of the sealing region comprises a non-peelable or permanent seal. The portion of the sealing region comprising a non-peelable or permanent seal is not designed to be opened by peeling like the peelable portion. The non-peelable or permanent seal may comprise part of the sealing region at which the first and second films are difficult to separate.

In some embodiments, the non-peelable portion of the sealing region between the first and second films comprises a non-peelable or permanent seal that is independently chosen from: a heat seal, an adhesive seal, a radio frequency seal, an ultrasonic seal, a laser seal, an induction seal, and a cold seal. In some embodiments, the non-peelable portion of the sealing region between the first and second films comprises a non-peelable or permanent seal that is independently selected from the group consisting of: a heat seal, an adhesive seal, a radio frequency seal, an ultrasonic seal, a laser seal, an induction seal, and a cold seal.

In some embodiments, the peelable and non-peelable portions of the sealing region comprise the same seal type. In other embodiments, the peelable and non-peelable portions of the sealing region comprise different seal types.

In preferred embodiments, the sealing region defines the perimeter of the container cavity. In such embodiments, the sealing region preferably has a top seal zone, a bottom seal zone, and opposed first and second side seal zones. The top and bottom seal zones are preferably opposed.

In some embodiments, the perimeter of the cavity defined by the sealing region is of a shape independently chosen from: a rectangle, a square, a circle, an oval, and arch and a trapezoid. In some embodiments, the perimeter of the cavity defined by the sealing region is of a shape independently selected from the group consisting of: a rectangle, a square, a circle, an oval, and arch and a trapezoid. One with skill in the art will recognize that the perimeter of the cavity may take a range of other shapes.

The entire sealing region preferably comprises a continuous seal. In such embodiments, the container is fully sealed prior to opening.

In some embodiments, at least a portion of the sealing region comprises a peripheral seal at or close to the edge of the first and second films.

In some embodiments, at least one seal zone extends along at least a portion of an edge of the first and second films. In some embodiments, the first side seal zone of the sealing region extends along at least a portion of an edge of the first and second films. The first side seal zone may be at or spaced from the edge of the first and second films along which the first side seal zone extends. In some embodiments, the second side seal zone of the sealing region extends along at least a portion of an edge of the first and second films. The second side seal zone may be at or spaced from the edge of the first and second films along which second side seal zone extends. In some embodiments, the first and second side seal zones extend along at least a portion of opposing edges of the first and second films. The first and second side seal zones may be at or spaced from the edges of the first and second films along which they extend.

In some embodiments, the top seal zone of the sealing region extends along at least a portion of an edge of the first and second films. The top seal zone may be at or spaced from the edge of the first and second films along which the top seal zone extends. In some embodiments, the bottom seal zone of the sealing region extends along at least a portion of an edge of the first and second films. The bottom seal zone may be at or spaced from the edge of the first and second films along which the bottom seal zone extends. In some embodiments, the top and bottom seal zones extend along at least a portion of opposing edges of the first and second films. The top and bottom seal zones may be at or spaced from the edges of the first and second films along which they extend.

In some embodiments, the first and second side seal zones extend along at least a portion of opposing edges of the first and second films, and the top and bottom seal zones extend along at least a portion of opposing edges of the first and second films. In such embodiments, at least one seal zone may be spaced from the edge of the first and second films along which said seal zone extends. In some embodiments, only one seal zone may be spaced from the edge of the first and second films along which said seal zone extends, and the other seal zones may be at the edges of the first and second films along which they extend. In some embodiments, the top seal zone is spaced from the edge of the first and second films along which the top seal zone extends, and the other seal zones may be at the edges of the first and second films along which the extend.

In some embodiments, at least a portion of at least one seal zone is peelable. At least a portion of the top seal zone or the entire top seal zone may be peelable. At least a portion of the first side seal zone or the entire first side seal zone may be peelable. At least a portion of the second side seal zone or the entire second side seal zone may be peelable. At least a portion of the bottom seal zone or the entire bottom seal zone may be peelable.

In preferred embodiments, at least a portion of the top seal zone comprises the peelable opening. In such embodiments, at least a portion of the top seal zone or the entire top seal zone is peelable. In some embodiments, only the top seal zone or a portion thereof is peelable and defines the peelable opening.

In preferred embodiments, the top seal zone and at least a portion of the first and second side seal zones adjacent to the top seal zone comprise the peelable opening. In such embodiments, the first and second films are at least partially separable adjacent to the top seal zone and peelable portions of the first and second side seal zones to open the container, in use, by peeling the films apart along the top seal zone and at least partially along the peelable portions of the first and second side seal zones of the sealing region. In such embodiments, the top seal zone and at least a portion of the first and second side seal zones adjacent to the top seal zone are peelable. In some embodiments, the top seal zone and the entire first and second side seal zones are peelable.

In some embodiments, the entire sealing region is peelable. In other embodiments, at least a portion of the sealing region is non-peelable and may comprise a non-peelable or permanent seal. In some embodiments, an end portion of the first and second side seal zones distal from the top seal zone is non-peelable.

In some embodiments, at least part of the bottom seal zone is non-peelable. The entire bottom seal zone may be non-peelable.

In some embodiments, an end portion of the first and second side seal zones distal from the top seal zone is non-peelable, and the bottom seal zone is non-peelable.

In some embodiments, the container comprises at least one peel-stop located in at least one seal zone of the sealing region, wherein the peel-stop resists, limits or prevents peeling of the first and second films. In some embodiments, the container comprises a first peel-stop located in a seal zone of the sealing region and a second peel-stop located in an opposed seal zone of the sealing region, wherein the first and second peel-stops resist, limit or prevent peeling of the first and second films past the peel-stops in the opposed side seal zones of the sealing region.

The first and second peel-stops may preferably be aligned. Alternatively, the first and second peel-stops may be offset. For example the first peel-stop may be lower than the second peel-stop, or vice versa.

In some embodiments, the container comprises a first peel-stop located in the first side seal zone of the sealing region and a second peel-stop located in the second side seal zone of the sealing region, wherein the first and second peel-stops resist, limit or prevent peeling of the first and second films past the peel-stops in the first and second side seal zones of the sealing region.

In some embodiments, at least one peel-stop, preferably the first and second peel-stops comprise seal zone portions that require more force to peel apart than other seal zone portions.

In some embodiments, at least one peel-stop, preferably the first and second peel-stops comprise widened seal zone portions or seal zone portions of greater area compared to other seal zone portions. In some embodiments, at least one peel-stop, preferably the first and second peel-stops comprise peel interrupt elements. The peel interrupt elements may comprise cuts or perforations through the first film and/or second film in the peel-stops. The cuts or perforations may be die-cuts or laser-cuts. The cuts or perforations may be present in a variety of shapes and sizes. In some embodiments, the peel-interrupt elements may be J-shaped, V-shaped, or hook-shaped. In some embodiments, if the user continues to peel the first and second films apart once the peel-interrupt elements have been reached, the peel-interrupt elements may direct the force toward the centre of the films which will prevent further peeling or make the films harder to peel apart. In some embodiments, at least one peel-stop, preferably the first and second peel-stops provide a tactile feel that alerts the user that the container has been sufficiently opened so that the user knows to stop peeling.

The first and second peel-stops may be positioned such that the peelable opening and/or container can be opened to expose a portion of the medical device. The first and second peel-stops may be positioned such that only an end of the medical device is exposed when the container is opened.

In embodiments wherein the container comprises a peel-stop located in the first side seal zone of the sealing region and a second peel-stop located in the second side seal zone of the sealing region, the first and second peel-stops may be positioned closer to the top seal zone of the sealing region than the bottom seal zone. The first and second peel-stops may be positioned such that half or less, or a third or less of the length of the medical device is exposed when the peelable opening and/or container is opened to the positions of the first and second peel-stops. Alternatively, the first and second peel-stops may be positioned closer to the bottom seal zone of the sealing region than the top seal zone.

In some embodiments, at least one seal zone may comprise a plurality of peel-stops. In some embodiments, opposed seal zones may both comprise greater than one peel-stop. In some embodiments, the opposed first and second side seal zones may both comprise greater than one peel-stop.

In some embodiments, the container comprises opening flaps formed by an unsealed region of the first and second films.

In some embodiments, at least one seal zone is set in from an edge of the first and second films and the opening flaps are formed by an unsealed region between the edge and the seal zone. The set-in seal zone is preferably at least partially peelable. In preferred embodiments, the top seal zone is set in from an edge of the first and second films and the opening flaps are formed by an unsealed region between the edge and the top seal zone.

In some embodiments, the opening flap of the first and/or second film comprises a roughened area on the outer surface thereof that facilitates easier gripping of the flap.

In some embodiments, the unsealed region of one of the films of the opening flaps is longer than the unsealed region of the film of the opening flaps. This allows the user to easily grasp the opening flap with the longer unsealed region and more easily separate the first and second films.

In some embodiments, the opening flaps comprise a cut-out formed through the first and/or second films in the unsealed region of the opening flaps. In some embodiments, the cut-out comprises a finger hole. In some embodiments, the finger hole has a rounded triangular shape. In embodiments wherein the opening flaps are formed by an unsealed region between an edge of the first and second films and the top seal zone, the finger hole may taper towards the top seal.

In some embodiments, the top seal zone may be in a linear configuration or a non-linear configuration. In some embodiments, the top seal zone has a pointed shape, which may comprise a chevron shape. The top seal zone may have a rounded chevron shape or an arch shape.

In some embodiments, the top seal zone comprises a pair of seal lines which continue from the first and second side seal zones and gradually curve to meet. The pair of seal lines may meet at a point. The pair of seal lines may meet at a midpoint.

In some embodiments, the container is a pouch. In some embodiments, the container is flexible. The container may be a flexible pouch.

In preferred embodiments, the medical device is an insertable medical device. The medical device may be independently chosen from: a cannula, a catheter, an endoscope, a syringe, and a stent. The medical device may be independently selected from the group consisting of: a cannula, a catheter, an endoscope, a syringe, and a stent.

In some embodiments, the medical device is a cannula or a catheter. The medical device may preferably be an intermittent catheter.

In preferred embodiments, the medical device is a urinary catheter. In preferred embodiments, the catheter is an intermittent urinary catheter. Such a catheter is typically inserted into a body for short time periods, such as less than a day. Alternatively, the catheter may be an indwelling (Foley) catheter. Such a catheter is typically inserted and kept in a body for long periods of time, such as several days to months.

The catheter may be a single use catheter or a multiple use catheter.

In some embodiments, the catheter comprises a hollow tubular body, preferably a hollow polymeric tubular body. The hollow polymeric tubular body may comprise a base polymer.

In some embodiments, the catheter further comprises at least one additive, preferably at least one lubricious additive. Preferably, the hollow tubular body comprises at least one additive.

In some embodiments, at least one additive is a hydrophilic or amphiphilic additive.

According to a third aspect of the invention, there is provided a packaged medical device comprising a medical device stored in a container, the container comprising: a first polymer film and a second polymer film, wherein the first and second polymer films are sealed together at a sealing region to form a cavity therebetween in which the medical device is contained, wherein the first and second films each have an inner surface that faces the inner surface of the other film and an outer surface that faces the exterior of the container, wherein the inner surface of the first film and the inner surface of the second film comprise a non-oriented polyolefin, and the outer surface of the first film and the outer surface of the second film comprise an oriented polyolefin, and wherein at least a portion of the sealing region is peelable to form an opening in the container.

The container of the third aspect of the invention is preferably the container of the first aspect of the invention. The medical device of the third aspect of the invention is preferably the medical device of the first aspect of the invention. In preferred embodiments, the medical device is a urinary catheter. In preferred embodiments, the catheter is an intermittent urinary catheter. Such a catheter is typically inserted into a body for short time periods, such as less than a day. Alternatively, the catheter may be an indwelling (Foley) catheter. Such a catheter is typically inserted and kept in a body for long periods of time, such as several days to months.

The catheter may be a single use catheter or a multiple use catheter. Statements of invention above relating to the first and second aspects of the invention may also be applied mutatis mutandis to the third aspect of the invention. Statements of invention for any other aspect of the invention may also be applied mutatis mutandis to the third aspect of the invention. Statements of invention below for the third aspect of the invention may also be applied mutatis mutandis to any other aspect of the invention.

In some embodiments, the container further comprises a medium stored therein. The medium may be independently chosen from: a sterilising agent, a wetting agent, a lubricating agent, and combinations thereof. The medium may be independently selected from the group consisting of: a sterilising agent, a wetting agent, a lubricating agent, and combinations thereof.

The medium may be a liquid, which may be a solution., or a gel The medium may be an aqueous solution or an aqueous gel. Aqueous solutions and gels are particularly effective, as water allows for optimal medical device surface lubricity, especially catheter surface lubricity.

The medium may be a catheter wetting agent. The medium may encourage hydrophilic portions of lubricating additives within the catheter to seek towards an outer surface of the catheter, which further enhances the lubricating effect of the additive.

In some embodiments, the medium is in direct contact with the medical device. The medium may be in direct contact with at least one surface of the medical device. The at least one surface may comprise an outer surface of the medical device. The medium may cover at least part of the outer surface of the medical device.

The at least one surface may comprise an inner surface of the medical device and the medium may cover at least part of the inner surface.

In some embodiments, the medical device is submerged in the medium. The medical device may be fully submerged in the medium. In such embodiments, the medium may comprise at least 10% of the internal volume of the container, or at least 20, 30, 40, 50, 60, 70, 80, 90, or at least 95% of the internal volume of the container.

In some embodiments, the medical device is not fully submerged in the medium. In such embodiments, the medium may comprise no greater than 30% of the internal volume of the container, or no greater than 25, 20, 15, 10, 5, 4, 3, 2, or no greater than 1% of the internal volume of the container. In such embodiments, the medium may be in direct contact with at least part of the medical device, preferably with at least one surface thereof. In some embodiments, the medium may be able to move freely within the container. In such embodiments, the medium may be configured to slosh around within the container, such as when the container is shaken and/or due to natural movement of the container by the user.

In some embodiments, the medium may not be in direct contact with the medical device. The medium may be contained in a separate medium container that is stored in the container of the invention. The medium container may be a bag or sachet. In some embodiments, the medium container is pierceable, in use, to release the contained medium from the medium container and into direct contact with the medical device in the container of the invention, preferably without requiring opening of the container of the invention.

In some embodiments, opening the container of the invention, in use, brings the medium into direct contact with the medical device. The medium container may be configured to rupture or break to release the medium upon opening of the container of the invention. In some embodiments, opening the container, in use, pierces the medium container and releases the contained medium from the medium container and into direct contact with the medical device.

In embodiments wherein the medical device is a catheter, the user may release the medium from the medium container and apply the medium to the outer surface of the catheter prior to opening the container of the invention, prior to removing the catheter from the container of the invention and/or prior to inserting the catheter.

In some preferred embodiments, there is provided a packaged medical device comprising a medical device stored in a container, the container comprising: a first polymer film and a second polymer film, wherein the first and second polymer films are sealed together at a sealing region to form a cavity therebetween in which the medical device is contained, wherein the first and second films each have an inner surface that faces the inner surface of the other film and an outer surface that faces the exterior of the container, wherein the inner surface of the first film and the inner surface of the second film comprise a non-oriented polyolefin, and the outer surface of the first film and the outer surface of the second film comprise an oriented polyolefin, and wherein at least a portion of the sealing region is peelable to form an opening in the container, and wherein the oriented polyolefin and/or the non-oriented polyolefin is a low-density polyolefin.

In some preferred embodiments, there is provided a packaged medical device comprising a medical device stored in a container, the container comprising: a first polymer film and a second polymer film, wherein the first and second polymer films are sealed together at a sealing region to form a cavity therebetween in which the medical device is contained, wherein the first and second films each have an inner surface that faces the inner surface of the other film and an outer surface that faces the exterior of the container, wherein the inner surface of the first film and the inner surface of the second film comprise a non-oriented polyolefin, and the outer surface of the first film and the outer surface of the second film comprise an oriented polyolefin, and wherein at least a portion of the sealing region is peelable to form an opening in the container, and wherein only the non-oriented polyolefin is a low-density polyolefin.

In some preferred embodiments, there is provided a packaged medical device comprising a medical device stored in a container, the container comprising: a first polymer film and a second polymer film, wherein the first and second polymer films are sealed together at a sealing region to form a cavity therebetween in which the medical device is contained, wherein the first and second films each have an inner surface that faces the inner surface of the other film and an outer surface that faces the exterior of the container, wherein the inner surface of the first film and the inner surface of the second film comprise a non-oriented polyolefin, and the outer surface of the first film and the outer surface of the second film comprise an oriented polyolefin, and wherein at least a portion of the sealing region is peelable to form an opening in the container, and wherein the oriented polyolefin and/or the non-oriented polyolefin is a linear low-density polyolefin.

In some preferred embodiments, there is provided a packaged medical device comprising a medical device stored in a container, the container comprising: a first polymer film and a second polymer film, wherein the first and second polymer films are sealed together at a sealing region to form a cavity therebetween in which the medical device is contained, wherein the first and second films each have an inner surface that faces the inner surface of the other film and an outer surface that faces the exterior of the container, wherein the inner surface of the first film and the inner surface of the second film comprise a non-oriented polyolefin, and the outer surface of the first film and the outer surface of the second film comprise an oriented polyolefin, and wherein at least a portion of the sealing region is peelable to form an opening in the container, and wherein only the non-oriented polyolefin is a linear low-density polyolefin.

In some preferred embodiments, there is provided a packaged medical device comprising a medical device stored in a container, the container comprising: a first polymer film and a second polymer film, wherein the first and second polymer films are sealed together at a sealing region to form a cavity therebetween in which the medical device is contained, wherein the first and second films each have an inner surface that faces the inner surface of the other film and an outer surface that faces the exterior of the container, wherein the inner surface of the first film and the inner surface of the second film comprise a non-oriented polyolefin, and the outer surface of the first film and the outer surface of the second film comprise an oriented polyolefin, and wherein at least a portion of the sealing region is peelable to form an opening in the container, and wherein the oriented polyolefin and/or the non-oriented polyolefin is polyethylene.

In some preferred embodiments, there is provided a packaged medical device comprising a medical device stored in a container, the container comprising: a first polymer film and a second polymer film, wherein the first and second polymer films are sealed together at a sealing region to form a cavity therebetween in which the medical device is contained, wherein the first and second films each have an inner surface that faces the inner surface of the other film and an outer surface that faces the exterior of the container, wherein the inner surface of the first film and the inner surface of the second film comprise a non-oriented polyolefin, and the outer surface of the first film and the outer surface of the second film comprise an oriented polyolefin, and wherein at least a portion of the sealing region is peelable to form an opening in the container, and wherein the oriented polyolefin and/or the non-oriented polyolefin is low-density polyethylene.

In some preferred embodiments, there is provided a packaged medical device comprising a medical device stored in a container, the container comprising: a first polymer film and a second polymer film, wherein the first and second polymer films are sealed together at a sealing region to form a cavity therebetween in which the medical device is contained, wherein the first and second films each have an inner surface that faces the inner surface of the other film and an outer surface that faces the exterior of the container, wherein the inner surface of the first film and the inner surface of the second film comprise a non-oriented polyolefin, and the outer surface of the first film and the outer surface of the second film comprise an oriented polyolefin, and wherein at least a portion of the sealing region is peelable to form an opening in the container, and wherein only the non-oriented polyolefin is low-density polyethylene.

In some preferred embodiments, there is provided a packaged medical device comprising a medical device stored in a container, the container comprising: a first polymer film and a second polymer film, wherein the first and second polymer films are sealed together at a sealing region to form a cavity therebetween in which the medical device is contained, wherein the first and second films each have an inner surface that faces the inner surface of the other film and an outer surface that faces the exterior of the container, wherein the inner surface of the first film and the inner surface of the second film comprise a non-oriented polyolefin, and the outer surface of the first film and the outer surface of the second film comprise an oriented polyolefin, and wherein at least a portion of the sealing region is peelable to form an opening in the container, and wherein the oriented polyolefin and/or the non-oriented polyolefin is linear low-density polyethylene.

In some preferred embodiments, there is provided a packaged medical device comprising a medical device stored in a container, the container comprising: a first polymer film and a second polymer film, wherein the first and second polymer films are sealed together at a sealing region to form a cavity therebetween in which the medical device is contained, wherein the first and second films each have an inner surface that faces the inner surface of the other film and an outer surface that faces the exterior of the container, wherein the inner surface of the first film and the inner surface of the second film comprise a non-oriented polyolefin, and the outer surface of the first film and the outer surface of the second film comprise an oriented polyolefin, and wherein at least a portion of the sealing region is peelable to form an opening in the container, and wherein only the non-oriented polyolefin is linear low-density polyethylene.

In some preferred embodiments, there is provided a packaged medical device comprising a medical device stored in a container, the container comprising: a first polymer film and a second polymer film, wherein the first and second polymer films are sealed together at a sealing region to form a cavity therebetween in which the medical device is contained, wherein the first and second films each have an inner surface that faces the inner surface of the other film and an outer surface that faces the exterior of the container, wherein the inner surface of the first film and the inner surface of the second film comprise a non-oriented polyolefin, and the outer surface of the first film and the outer surface of the second film comprise an oriented polyolefin, and wherein at least a portion of the sealing region is peelable to form an opening in the container, and wherein the oriented polyolefin and the non-oriented polyolefin comprise the same polyolefin.

In some preferred embodiments, there is provided a packaged medical device comprising a medical device stored in a container, the container comprising: a first polymer film and a second polymer film, wherein the first and second polymer films are sealed together at a sealing region to form a cavity therebetween in which the medical device is contained, wherein the first and second films each have an inner surface that faces the inner surface of the other film and an outer surface that faces the exterior of the container, wherein the inner surface of the first film and the inner surface of the second film comprise a non-oriented polyolefin, and the outer surface of the first film and the outer surface of the second film comprise an oriented polyolefin, and wherein at least a portion of the sealing region is peelable to form an opening in the container, and wherein the first film and/or the second film is made of a single polyolefin.

According to a fourth aspect of the invention, there is provided a method of manufacturing a container for storing a medical device, the method comprising the steps of:
(a) Providing a first polymer film and a second polymer film, the first and second polymer films each comprising a surface comprising an oriented polyolefin and an opposing surface comprising a non-oriented polyolefin;
(b) Providing a medical device; and
(c) Sealing the first and second polymer films together at a sealing region to form a cavity therebetween, wherein the non-oriented polyolefin surface of the first polymer film faces the non-oriented polyolefin surface of the second polymer film, and wherein at least a portion of the sealing region is peelable to form an opening in the container;
   wherein the medical device is inserted into the cavity before the sealing region is fully sealed.

The container of the fourth aspect of the invention is preferably the container of the first aspect of the invention. Statements of invention above relating to the first, second and third aspects of the invention may also be applied mutatis mutandis to the fourth aspect of the invention. Statements of invention below relating to the fourth aspect of the invention may also be applied mutatis mutandis to the other aspects of the invention.

In some embodiments, the medical device is placed between the first and second films before step (c), and the first and second seals are sealed around the medical device to form the cavity and contain the medical device in the cavity of the container.

In some embodiments, the first and second films are partially sealed together to partially form the cavity, the medical device is inserted into the partially formed cavity, and then the sealing region is fully sealed. In such embodiments, the first and second films may be partially sealed together to leave a slot or opening through which the medical device may be inserted into the cavity.

Step (c) may comprise sealing the first and second polymer films together using a method independently chosen from: heat sealing, cold sealing, adhesive sealing, cohesive sealing, weld sealing, sonic welding, ultrasonic welding, hot melt sealing, pressure-sensitive sealing, micro-embossed structure sealing, radio frequency welding, induction sealing, hot bar sealing, laser sealing, and combinations thereof. Step (c) may comprise sealing the first and second polymer films together using a method independently selected from the group consisting of: heat sealing, cold sealing, adhesive sealing, cohesive sealing, weld sealing, sonic welding, ultrasonic welding, hot melt sealing, pressure-sensitive sealing, micro-embossed structure sealing, radio frequency welding, induction sealing, hot bar sealing, laser sealing, and combinations thereof.

Step (c) may comprise sealing the first and second polymer films together at peelable portions of the sealing region using a method independently chosen from: heat sealing, cold sealing, adhesive sealing, cohesive sealing, weld sealing, ultrasonic welding, hot melt sealing, pressure-sensitive sealing, micro-embossed structure sealing, and combinations thereof. Step (c) may comprise sealing the first and second polymer films together at peelable portions of the sealing region using a method independently selected from the group consisting of: heat sealing, cold sealing, adhesive sealing, cohesive sealing, weld sealing, ultrasonic welding, hot melt sealing, pressure-sensitive sealing, micro-embossed structure sealing, and combinations thereof. In some preferred embodiments, step (c) comprises sealing the first and second polymer films together at peelable portions of the sealing region using heat sealing and/or cold sealing. In some preferred embodiments, step (c) comprises sealing the first and second films together at peelable portions of the sealing region using heat sealing.

At least a portion of the sealing region may be non-peelable and may comprise a non-peelable or permanent seal, preferably as described for the first aspect of the invention above. In some embodiments, step (c) comprises sealing the first and second polymer films together at the non-peelable portions of the sealing region using a method independently chosen from: heat sealing, adhesive sealing, radio frequency sealing, ultrasonic welding, laser sealing, induction sealing, cold sealing, and combinations thereof. In some embodiments, step (c) comprises sealing the first and second polymer films together at the non-peelable portions of the sealing region using a method independently selected from the group consisting of: heat sealing, adhesive sealing, radio frequency sealing, ultrasonic welding, laser sealing, induction sealing, cold sealing, and combinations thereof.

In some embodiments, step (c) comprises sealing the first and second polymer films together at the peelable portions and non-peelable portions of the sealing region using the same sealing method. Step (c) may comprise sealing the first and second polymer films together at the peelable portions using a lower sealing temperature than at the non-peelable portions. Step (c) may comprise sealing the first and second films together at the peelable portions using a lower sealing pressure than at the non-peelable portion. In some embodiments, step (c) comprises sealing the first and second polymer films together at the peelable portions and non-peelable portions of the sealing region using heat sealing and/or cold sealing. In some preferred embodiments, step (c) comprises sealing the first and second polymer films together at the peelable and non-peelable portions of the sealing region using heat sealing. Step (c) may comprise heat sealing the first and second polymer films together at the peelable portions using a lower sealing temperature than at the non-peelable portions. Step (c) may comprise heat sealing the first and second films together at the peelable portions using a lower sealing pressure than at the non-peelable portion.

In some embodiments, the method may comprise heat sealing the first and second films together at the sealing region at a sealing temperature of at least 80° C., or at least 85, or at least 90° C. The method may comprise heat sealing the first and second films together at the sealing region at a sealing temperature of no greater than 350° C., or no greater than 340, 330, 320, 310, 300, 290, 280, 270, 260, 250, 240, 230, 220, 210, 200, 190, 180, 170, 160, 150, or no greater than 145° C. The method may comprise heat sealing the first and second films together at the sealing region at a sealing temperature of between 80-300° C., or between 80-250, 80-200, 80-155, 85-150, or between 90-145° C.

In some embodiments, the method may comprise heat sealing the first and second films together at peelable portions of the sealing region using a sealing temperature of between 80-155° C., or between 85-150, or between 90-145° C.; and heat sealing the first and second films together at non-peelable portions of the sealing region using a higher sealing temperature, which may be between 145-300° C., or between 150-250° C.

In some preferred embodiments, there is provided a method of manufacturing a container for storing a medical device, the method comprising the steps of:
(a) Providing a first polymer film and a second polymer film, the first and second polymer films each comprising a surface comprising an oriented polyolefin and an opposing surface comprising a non-oriented polyolefin, wherein the oriented polyolefin and/or the non-oriented polyolefin is a low-density polyolefin;
(b) Providing a medical device; and
(c) Sealing the first and second polymer films together at a sealing region to form a cavity therebetween, wherein the non-oriented polyolefin surface of the first polymer film faces the non-oriented polyolefin surface of the second polymer film, and wherein at least a portion of the sealing region is peelable to form an opening in the container;
   wherein the medical device is inserted into the cavity before the sealing region is fully sealed.

In some preferred embodiments, there is provided a method of manufacturing a container for storing a medical device, the method comprising the steps of:
(a) Providing a first polymer film and a second polymer film, the first and second polymer films each comprising a surface comprising an oriented polyolefin and an opposing surface comprising a non-oriented polyolefin, wherein only the non-oriented polyolefin is a low-density polyolefin;
(b) Providing a medical device; and
(c) Sealing the first and second polymer films together at a sealing region to form a cavity therebetween, wherein the non-oriented polyolefin surface of the first polymer film faces the non-oriented polyolefin surface of the second polymer film, and wherein at least a portion of the sealing region is peelable to form an opening in the container;
   wherein the medical device is inserted into the cavity before the sealing region is fully sealed.

In some preferred embodiments, there is provided a method of manufacturing a container for storing a medical device, the method comprising the steps of:
(a) Providing a first polymer film and a second polymer film, the first and second polymer films each comprising a surface comprising an oriented polyolefin and an opposing surface comprising a non-oriented polyolefin, wherein the oriented polyolefin and/or the non-oriented polyolefin is a linear low-density polyolefin;
(b) Providing a medical device; and
(c) Sealing the first and second polymer films together at a sealing region to form a cavity therebetween, wherein the non-oriented polyolefin surface of the first polymer film faces the non-oriented polyolefin surface of the second polymer film, and wherein at least a portion of the sealing region is peelable to form an opening in the container;
   wherein the medical device is inserted into the cavity before the sealing region is fully sealed.

In some preferred embodiments, there is provided a method of manufacturing a container for storing a medical device, the method comprising the steps of:
(a) Providing a first polymer film and a second polymer film, the first and second polymer films each comprising a surface comprising an oriented polyolefin and an opposing surface comprising a non-oriented polyolefin, wherein only the non-oriented polyolefin is a linear low-density polyolefin;
(b) Providing a medical device; and
(c) Sealing the first and second polymer films together at a sealing region to form a cavity therebetween, wherein the non-oriented polyolefin surface of the first polymer film faces the non-oriented polyolefin surface of the second polymer film, and wherein at least a portion of the sealing region is peelable to form an opening in the container;
   wherein the medical device is inserted into the cavity before the sealing region is fully sealed.

In some preferred embodiments, there is provided a method of manufacturing a container for storing a medical device, the method comprising the steps of:
(a) Providing a first polymer film and a second polymer film, the first and second polymer films each comprising a surface comprising an oriented polyolefin and an opposing surface comprising a non-oriented polyolefin, wherein the oriented polyolefin and/or the non-oriented polyolefin is polyethylene;
(b) Providing a medical device; and
(c) Sealing the first and second polymer films together at a sealing region to form a cavity therebetween, wherein the non-oriented polyolefin surface of the first polymer film faces the non-oriented polyolefin surface of the second polymer film, and wherein at least a portion of the sealing region is peelable to form an opening in the container;
   wherein the medical device is inserted into the cavity before the sealing region is fully sealed.

In some preferred embodiments, there is provided a method of manufacturing a container for storing a medical device, the method comprising the steps of:
(a) Providing a first polymer film and a second polymer film, the first and second polymer films each comprising a surface comprising an oriented polyolefin and an opposing surface comprising a non-oriented polyolefin, wherein the oriented polyolefin and/or the non-oriented polyolefin is low-density polyethylene;
(b) Providing a medical device; and
(c) Sealing the first and second polymer films together at a sealing region to form a cavity therebetween, wherein the non-oriented polyolefin surface of the first polymer film faces the non-oriented polyolefin surface of the second polymer film, and wherein at least a portion of the sealing region is peelable to form an opening in the container;
   wherein the medical device is inserted into the cavity before the sealing region is fully sealed.

In some preferred embodiments, there is provided a method of manufacturing a container for storing a medical device, the method comprising the steps of:
(a) Providing a first polymer film and a second polymer film, the first and second polymer films each comprising a surface comprising an oriented polyolefin and an opposing surface comprising a non-oriented polyolefin, wherein only the non-oriented polyolefin is low-density polyethylene;
(b) Providing a medical device; and
(c) Sealing the first and second polymer films together at a sealing region to form a cavity therebetween, wherein the non-oriented polyolefin surface of the first polymer film faces the non-oriented polyolefin surface of the second polymer film, and wherein at least a portion of the sealing region is peelable to form an opening in the container;
   wherein the medical device is inserted into the cavity before the sealing region is fully sealed.

In some preferred embodiments, there is provided a method of manufacturing a container for storing a medical device, the method comprising the steps of:
(a) Providing a first polymer film and a second polymer film, the first and second polymer films each comprising a surface comprising an oriented polyolefin and an opposing surface comprising a non-oriented polyolefin, wherein the oriented polyolefin and/or the non-oriented polyolefin is linear low-density polyethylene;
(b) Providing a medical device; and
(c) Sealing the first and second polymer films together at a sealing region to form a cavity therebetween, wherein the non-oriented polyolefin surface of the first polymer film faces the non-oriented polyolefin surface of the second polymer film, and wherein at least a portion of the sealing region is peelable to form an opening in the container;
   wherein the medical device is inserted into the cavity before the sealing region is fully sealed.

In some preferred embodiments, there is provided a method of manufacturing a container for storing a medical device, the method comprising the steps of:
(a) Providing a first polymer film and a second polymer film, the first and second polymer films each comprising a surface comprising an oriented polyolefin and an opposing surface comprising a non-oriented polyolefin, wherein only the non-oriented polyolefin is linear low-density polyethylene;
(b) Providing a medical device; and
(c) Sealing the first and second polymer films together at a sealing region to form a cavity therebetween, wherein the non-oriented polyolefin surface of the first polymer film faces the non-oriented polyolefin surface of the second polymer film, and wherein at least a portion of the sealing region is peelable to form an opening in the container;
   wherein the medical device is inserted into the cavity before the sealing region is fully sealed.

### DETAILED DESCRIPTION OF THE INVENTION

In order that the invention may be more clearly understood an embodiment thereof will now be described, by way of example only, with reference to the accompanying drawings, of which:

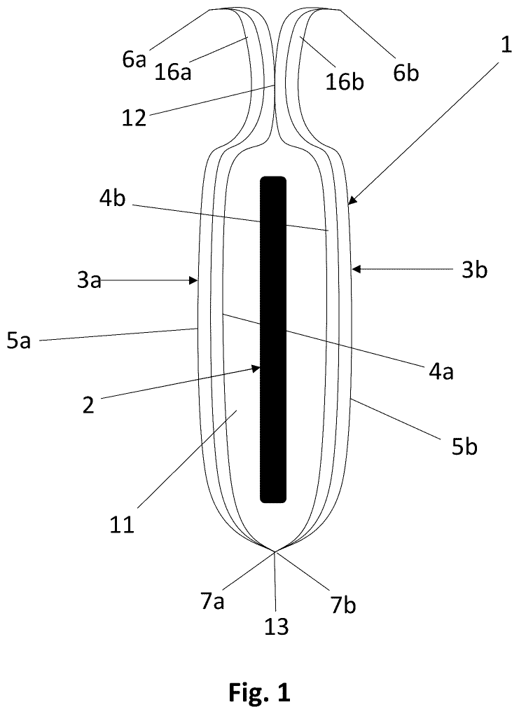

FIG. 1 is a side cross-sectional view of an embodiment of a container of the invention shown in a fully sealed configuration.

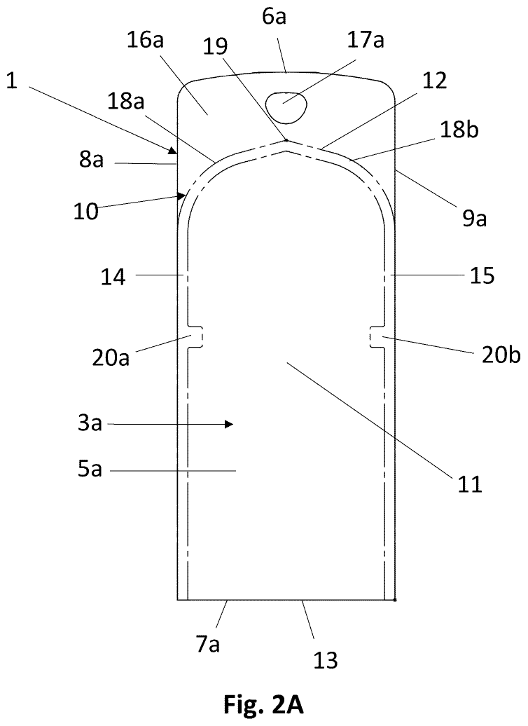

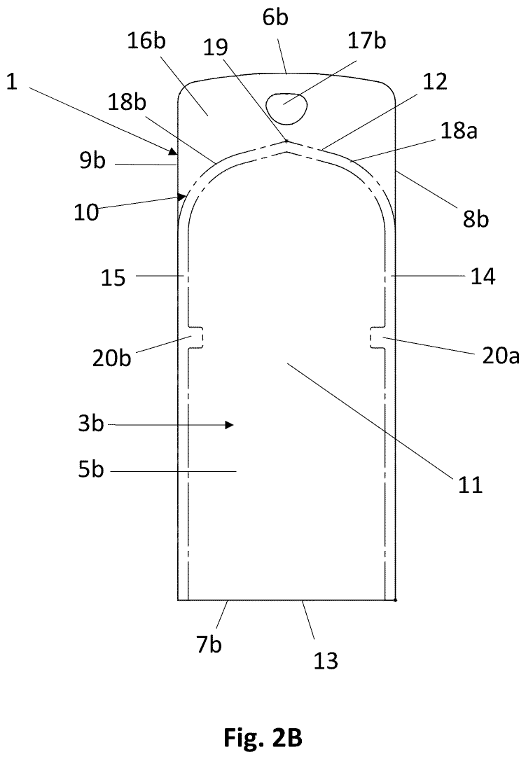

FIG. 2A is a top-down view of the container of FIG. 1 in a fully sealed configuration showing the outer surface of the first film of the container.

FIG. 2B is another top-down view of the container of FIG. 1 in a fully sealed configuration showing the outer surface of the second film of the container.

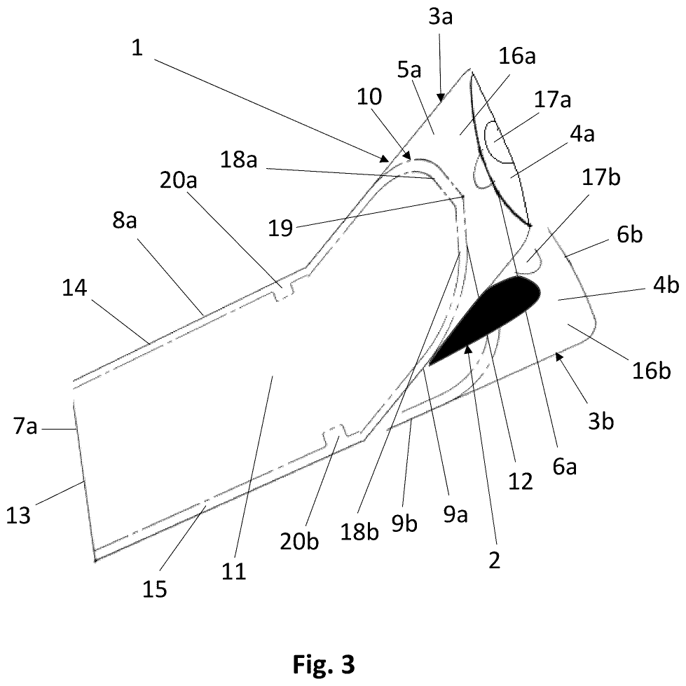

FIG. 3 is a perspective view of the container of FIG. 1 shown in a partially open configuration.

FIGS. 1, 2A, 2B and 3 illustrate an embodiment of a container 1 of the invention for storing a medical device, which in this embodiment is an intermittent urinary catheter 2. The container takes the form of pouch 1. Pouch 1 comprises a first film 3a and a second film 3b.

Referring to FIGS. 1 and 2A, the first film 3a is a rectangular two-layer laminated polymer film comprising an inner surface 4a, an outer surface 5a, a top edge 6a, a bottom edge 7a, and opposed first and second side edges 8a and 9a. The inner surface 4a comprises a separate layer of the laminated film 3a to the outer surface 5a. The layer of the film 3a comprising the inner surface 4a is laminated to the layer of the film 3a comprising the outer surface 5a by an adhesive used to bond the two layers together. The inner surface 4a is made of non-oriented linear low-density polyethylene and the outer surface 5a is made of biaxially oriented polyethylene.

Referring to FIGS. 1 and 2B, the second film 3b is also a rectangular two-layer laminated polymer film comprising an inner surface 4b, an outer surface 5b, a top edge 6b, a bottom edge 7b, and opposed first and second side edges 8b and 9b. The second film 3b comprises the same laminated film as the first film 3a. Similarly, the inner surface 4b of the second film 3b is made of non-oriented linear low-density polyethylene and the outer surface 5b of the second film 3b is made of biaxially oriented polyethylene.

As shown in FIG. 1, the inner surface 4a of the first film 3a faces the inner surface 4b of the second film 3b. The outer surfaces 5a and 5b of the first and second films 3a and 3b face the exterior of the pouch 1.

In the sealed configuration of the pouch 1 (FIGS. 1, 2A and 2B), the inner surface 4a of the first film 3a is sealed to the inner surface 4b of the second film 3b at a sealing region 10 to define a three-dimensional cavity 11 between the first film 3a and the second film 3b. The medical device 2 is contained within the cavity 11 formed between the two films 3a and 3b. When sealed, the pouch 1 maintains a sterile internal environment within the cavity 11.

The seal formed at the sealing region 10 is a continuous seal. The sealing region 10 comprises a top seal zone 12, a bottom seal zone 13, and opposed first and second side seal zones 14 and 15. The first side seal zone 14 extends along a portion of the first side edges 8a and 8b of the first and second films 3a and 3b, and the second side seal zone 15 extends along a portion of the second side edges 9a and 9b of the first and second films 3a and 3b. The first and second side seal zones 14 and 15 are at the first and second side edges 8a, 8b, 9a, and 9b of the first and second films 3a and 3b. The bottom seal zone 13 of the sealing region 10 extends along the bottom edges 7a and 7b of the first and second films 3a and 3b. The bottom seal zone 13 is at the bottom edges 7a and 7b of the first and second films 3a and 3b.

The top seal zone 12 of the sealing region 10 is set in from the top edges 6a and 6b of the first and second films 3a and 3b, such that unsealed regions 16a and 16b are formed between the top edges 6a and 6b of the first and second films 3a and 3b. The unsealed regions 16a and 16b of the first and second films 3a and 3b define a pair of opening flaps. The unsealed regions 16a and 16b of the first and second films 3a and 3b comprise cut-outs formed through the first and second films 3a and 3b in the unsealed regions 16a and 16b, to aid a user in grasping the films 3a and 3b. The cut-outs take the form of finger holes 17a and 17b. The finger holes 17a and 17b have a rounded triangular shape and taper towards the top seal zone 12 of the sealing region 10.

The top seal zone 12 of the sealing region 10 comprises a pair of seal lines 18a and 18b which continue from the first and second side seal zones 14 and 15 and gradually curve to meet at a midpoint 19.

The seal formed at the sealing region 10 is a heat seal. The heat seal formed at the top seal zone 12 and first and second side seal zones 14 and 15 of the sealing region 10 comprises a peelable seal that allows the first film 3a and the second film 3b to be peeled apart, in use, to form an opening in the pouch 1, as displayed in FIG. 3. The heat seal formed at the bottom seal zone 13 of the sealing region 10 comprises a non-peelable seal at which the first and second films 3a and 3b are difficult and not intended to peel apart.

The pouch 1 also includes a first peel-stop 20a located in the first side seal zone 14 of the sealing region 10, and a second peel-stop 20b located in the second side seal zone 15 of the sealing region 10. The first and second peel-stops 20a and 20b are aligned with each other and are positioned closer to the top seal zone 12 of the sealing region 10 than the bottom seal zone 13. The first and second peel-stops 20a and 20b resist, limit or prevent peeling apart of the first and second films 3a and 3b past the peel-stops 20a and 20b in the first and second side seal zones 14 and 15 of the sealing region. The peel-stops 20a and 20b comprise widened seal zone portions that are of greater area compared to other portions of the first and second side seal zones 14 and 15. The peel-stops 20a and 20b comprise seal zone portions where more force is required to peel the first and second films 3a and 3b apart compared to other portions of the first and second side seal zones 14 and 15.

In use, the user opens the pouch 1 by grasping the first and second films 3a and 3b at the opening flaps defined by the unsealed regions 16a and 16b of the first and second films 3a and 3b. The user may insert their fingers through the finger holes 17a and 17b to aid in grasping the sheets. The user then pulls the first and second films 3a and 3b apart to initiate separation of the films 3a and 3b. As the first and second films 3a and 3b are pulled apart, the first and second films 3a and 3b peel apart firstly along the peelable top seal zone 12 of the sealing region 10 and then along the peelable first and second side seal zones 14 and 15 of the sealing region 10, as shown in FIG. 3. As the user continues to pull the first and second films 3a and 3b apart, the films 3a and 3b continue to peel apart along the peelable first and second side seal zones 14 and 15, as shown in FIG. 3, until reaching the peel-stops 20a and 20b. When the peel-stops 20a and 20b are reached, the peel-stops 20a and 20b provide a tactile feel that alerts the user that the pouch 1 has been sufficiently opened so that the user knows to stop peeling. The peel-stops 20a and 20b are positioned such that when the pouch is opened up to this point, a selected portion of the contained medical device 2 is exposed and the user may grasp the medical device 2 and remove it from the pouch 1.

The combination of the non-oriented polyolefin inner surfaces (4a and 4b) (non-oriented linear low-density polyethylene) and the oriented polyolefin outer surfaces (5a and 5b) (biaxially oriented polyethylene) of the first and second films provides for optimal sealing conditions, which allows for a strong and effective seal to be formed, which allows for the sterility of the contents of the container to be maintained when closed. Moreover, such film materials also allow for a peelable portion of the sealing region 10 that is easy to peel, without the films 3a and 3b sticking and without requiring a large amount of force to be imparted.

The film materials also allow for selective sealing together of the inner surfaces (4a and 4b) of the films. The outer surfaces (5a and 5b) are minimally impacted by the sealing process. The result is a pouch with optimal mechanical properties and which suffers minimally from undesired tearing.

Overall, the combination of these film materials and their properties enables the provision of a container that both provides a sterile internal environment and that is easy to open, making it well-suited for storing medical devices, particularly catheters, in a safe and accessible manner.

Further advantageously, the pouch 1 is also fully recyclable and ready for polyethylene recycling streams without any modification required.

The above embodiments are described by way of example only. Many variations are possible without departing from the scope of the invention, as defined in the appended claims.

1. An intermittent catheter pouch for storing an intermittent catheter, the pouch comprising: a first polymer film and a second polymer film, wherein the first and second polymer films are sealed together at a sealing region to form a cavity therebetween, wherein the first and second films each have an inner surface that faces the inner surface of the other film and an outer surface that faces the exterior of the pouch, wherein the inner surface of the first film and the inner surface of the second film comprise a non-oriented polyolefin, and the outer surface of the first film and the outer surface of the second film comprise an oriented polyolefin, and wherein at least a portion of the sealing region is peelable to form an opening in the pouch.

2. An intermittent catheter pouch as claimed in claim 1, wherein the inner surface of the first film comprises the same polyolefin as the inner surface of the second film.

3. An intermittent catheter as claimed in any preceding claim, wherein the first film and/or the second film is made of a single polyolefin.

4. An intermittent catheter pouch as claimed in any preceding claim, wherein the oriented polyolefin and/or non-oriented polyolefin is a thermoplastic polyolefin.

5. An intermittent catheter pouch as claimed in any preceding claim, wherein the oriented polyolefin and the non-oriented polyolefin comprise the same polyolefin.

6. An intermittent catheter pouch as claimed in any preceding claim, wherein the oriented polyolefin and/or the non-oriented polyolefin, preferably only the non-oriented polyolefin, is a low-density polyolefin.

7. An intermittent catheter pouch as claimed in any preceding claim, wherein the oriented polyolefin and/or the non-oriented polyolefin, preferably only the non-oriented polyolefin, is a linear low-density polyolefin.

8. An intermittent catheter pouch as claimed in any preceding claim, wherein the oriented polyolefin and/or the non-oriented polyolefin is independently chosen from: polyethylene, polypropylene, ethylene-vinyl acetate, polybutene, an ethylene-alpha olefin copolymer, ethylene methyl acrylate, polyvinyl alcohol, polyvinyl chloride, polyvinylidene chloride, ethylene vinyl alcohol, acrylonitrile-butadiene-styrene, poly(methyl methacrylate), polystyrene, polymethylpentene, polyvinyl acetate, polyacrylamide, polyacrylate, ethylene/methacrylate copolymer, ethylene/n-butyl acrylate, ethylene/vinyl acetate/carbon monoxide, ethylene/acrylic acid, ethylene/methacrylic acid, and combinations, blends or copolymers thereof, and wherein the oriented polyolefin and/or the non-oriented polyolefin is preferably independently chosen from: polyethylene, polypropylene, and combinations, blends or copolymers thereof.

9. An intermittent catheter pouch as claimed in claim 8, wherein the oriented polyolefin and/or the non-oriented polyolefin is polyethylene.

10. An intermittent catheter pouch as claimed in claim 9, wherein the oriented polyolefin and/or the non-oriented polyolefin, preferably only the non-oriented polyolefin, is low-density polyethylene.

11. An intermittent catheter pouch as claimed in claim 9 or 10, wherein the oriented polyolefin and/or the non-oriented polyolefin, preferably only the non-oriented polyolefin, is linear low-density polyethylene.

12. An intermittent catheter pouch as claimed in any preceding claim, the oriented polyolefin is independently chosen from: a monoaxially oriented polyolefin, a biaxially oriented polyolefin, an oriented high-density polyolefin, an oriented ultra-high-molecular-weight polyolefin, a machine direction oriented polyolefin, and a tenter frame oriented polyolefin, and wherein the oriented polyolefin is preferably a biaxially oriented polyolefin.

13. An intermittent catheter pouch as claimed in any preceding claim, wherein the first film and/or second film is a laminate film formed of at least two polymer layers, wherein the inner and outer surfaces of the film preferably comprise different layers of the laminate film.

14. An intermittent catheter pouch as claimed in claim 13, wherein the first and second films both comprise the same laminate film.

15. An intermittent catheter pouch as claimed in any preceding claim, wherein the first and/or second film has a thickness of between 100-130 microns.

16. An intermittent catheter pouch as claimed in any preceding claim, wherein the peelable portion of the sealing region between the first and second films comprises a seal that is independently chosen from: a heat seal, a cold seal, an adhesive seal, a cohesive seal, a weld seal, an ultrasonic seal, a hot melt seal, a pressure-sensitive seal, and a seal created by a micro-embossed structure.

17. An intermittent catheter pouch as claimed in any preceding claim, wherein the entire sealing region is peelable.

18. An intermittent catheter pouch as claimed in any preceding claim, wherein the sealing region defines the perimeter of the pouch cavity, the sealing region having a top seal zone, a bottom seal zone, and opposed first and second side seal zones.

19. An intermittent catheter pouch as claimed in claim 18, wherein the top seal zone and at least a portion of the first and second side seal zones adjacent to the top seal zone comprise the peelable opening, and the first and second films are at least partially separable adjacent to the top seal zone and peelable portions of the first and second side seal zones to open the pouch, in use, by peeling the films apart along the top seal zone and at least partially along the peelable portions of the first and second side seal zones of the sealing region.

20. An intermittent catheter pouch as claimed in claim 19, wherein the pouch comprises a first peel-stop located in the first side seal zone of the sealing region and a second peel-stop located in the second side seal zone of the sealing region, wherein the first and second peel-stops resist, limit or prevent peeling of the first and second films past the peel-stops in the first and second side seal zones of the sealing region.

21. An intermittent catheter pouch as claimed in any preceding claim, wherein the pouch comprises opening flaps formed by an unsealed region of the first and second films.

22. An intermittent catheter pouch as claimed in claim 21 when dependent on any one of claims 18 to 20, wherein the top seal zone is set in from an edge of the first and second films and wherein the opening flaps are formed by an unsealed region between the edge and the top seal zone.

23. An intermittent catheter pouch as claimed in claim 21 or 22, wherein the opening flaps comprise a cut-out formed through the first and/or second films in the unsealed region of the opening flaps, wherein the cut-out preferably comprises a finger hole.

24. A packaged medical device comprising a medical device stored in a container, the container comprising: a first polymer film and a second polymer film, wherein the first and second polymer films are sealed together at a sealing region to form a cavity therebetween in which the medical device is contained, wherein the first and second films each have an inner surface that faces the inner surface of the other film and an outer surface that faces the exterior of the container, wherein the inner surface of the first film and the inner surface of the second film comprise a non-oriented polyolefin, and the outer surface of the first film and the outer surface of the second film comprise an oriented polyolefin, and wherein at least a portion of the sealing region is peelable to form an opening in the container.

25. A packaged medical device as claimed in claim 24, wherein the medical device is a cannula or a catheter, and preferably a urinary catheter.

26. A method of manufacturing a container comprising the steps of:
a. Providing a first polymer film and a second polymer film, the first and second polymer films each comprising a surface comprising an oriented polyolefin and an opposing surface comprising a non-oriented polyolefin;
b. Providing a medical device; and
c. Sealing the first and second polymer films together at a sealing region to form a cavity therebetween, wherein the non-oriented polyolefin surface of the first polymer film faces the non-oriented polyolefin surface of the second polymer film, and wherein at least a portion of the sealing region is peelable to form an opening in the container;
wherein the medical device is inserted into the cavity before the sealing region is fully sealed.
